(19) United States
(12) Patent Application Publication
(10) Pub. No.: US 2012/0009166 A1
(43) Pub. Date: Jan. 12, 2012

(54) Isolated monocyte populations and related therapeutic applications

(75) Inventors: Martin Friedlander, Del Mar, CA (US); Matthew R. Ritter, Oceanside, CA (US); Stacey K. Moreno, Lakeside, CA (US); Mohammad A. El-Kalay, Carlsbad, CA (US)

(73) Assignee: The Scripps Research Institute, La Jolla, CA (US)

(21) Appl. No.: 13/136,940

(22) Filed: Aug. 15, 2011

Related U.S. Application Data
(63) Continuation of application No. PCT/US2010/000477, filed on Feb. 19, 2010.
Application No. PCT/US2010/000477 is a continuation-in-part of application No. 12/658,440, filed on Feb. 5, 2010.
Application No. 12/658,440 is a division of application No. 11/600,895, filed on Nov. 16, 2006, now abandoned.
Application No. 11/600,895 is a continuation-in-part of application No. PCT/US2006/006411, filed on Feb. 24, 2006.
(60) Provisional application No. 61/208,173, filed on Feb. 20, 2009.
(60) Provisional application No. 61/283,244, filed on Nov. 30, 2009.
(60) Provisional application No. 60/656,037, filed on Feb. 24, 2005.

(51) Int. Cl. A61K 35/28; A61K 35/12; G01N 33/566; A61P 27/02; A61P 9/00; A61P 27/06; C12N 5/0786; A61K 35/14

(52) U.S. Cl. 424/93.71; 435/325; 435/372; 435/352; 435/375; 435/7.24

(57) ABSTRACT

The invention provides methods of using isolated monocyte populations to treat subjects suffering from various ocular vascular disease or ocular degenerative disorders. The present invention also provides novel methods for isolating substantially pure monocyte populations. The methods involve extracting a blood sample or a bone marrow sample from a subject, debulking red blood cells from the sample, and then separating remaining red blood cells and other cell types in the sample from monocytes. Instead of using any selection or labeling agents, the red blood cells and other cell types are separated from monocytes based on their size, granularity or density. The isolated monocytes can be further activated in vitro or ex vivo prior to being administered to a subject. Isolated cell populations containing substantially pure CD14⁺/CD33⁺ monocytes are also provided in the invention.

### CROSS-REFERENCE TO RELATED APPLICATIONS

The subject patent application is a continuation application under 35 U.S.C. 111(a) of international application PCT/US2010/000477 (filed Feb. 19, 2010), which in turn claims the benefit of priority to U.S. Provisional Patent Application Nos. 61/208,173 (filed Feb. 20, 2009) and 61/283,244 (Filed Nov. 30, 2009). The subject patent application is also a continuation-in-part application under 35 U.S.C. 120 of U.S. patent application Ser. No. 12/658,440 (filed Feb. 5, 2010), which is a divisional application of U.S. patent application Ser. No. 11/600,895 (filed Nov. 16, 2006), which is a continuation-in-part of International Application for Patent Serial No. PCT/US2006/006411 (filed Feb. 24, 2006), which claims the benefit of priority to U.S. Provisional Patent Application No. 60/656,037 (filed on Feb. 24, 2005). The full disclosures of the aforementioned priority applications are incorporated herein by reference in their entirety and for all purposes.

### STATEMENT OF GOVERNMENT SUPPORT

This invention was made with government support under Grant Nos. EY011254, EY014174 and EY017540 awarded by the National Institutes of Health. The government has certain rights in the invention.

### BACKGROUND OF THE INVENTION

Ocular vascular diseases such as age related macular degeneration (ARMD) and diabetic retinopathy (DR) are due to abnormal choroidal or retinal neovascularization respectively. They are the leading causes of visual loss in industrialized nations. Since the retina consists of well-defined layers of neuronal, glial, and vascular elements, relatively small disturbances such as those seen in vascular proliferation or edema can lead to significant loss of visual function. Inherited retinal degenerations, such as Retinitis Pigmentosa (RP), are also associated with vascular abnormalities, such as arteriolar narrowing and vascular atrophy. They affect as many as 1 in 3500 individuals and are characterized by progressive night blindness, visual field loss, optic nerve atrophy, arteriolar attenuation, and central loss of vision often progressing to complete blindness. While significant progress has been made in identifying factors that promote and inhibit angiogenesis, there are still no effective treatments to slow or reverse the progression of these retinal degenerative diseases.

There is a need in the art for better means for treating and preventing various ocular vascular diseases. The present invention is directed to this and other needs.

### SUMMARY OF THE INVENTION

In one aspect, the present invention provides isolated cell populations containing substantially pure monocytes that express CD33 antigen and CD14 antigen. Some of these isolated cell populations are isolated from a mammalian peripheral blood sample, a cord blood sample or a bone marrow sample. Some of the isolated cell populations are comprised of human cells or murine cells. In some of the isolated cell populations, at least 70%, 80% or 90% of the cells express surface markers CD14 and CD33. Some of the isolated cell populations do not contain cells that express CD34. Some of isolated cell populations are substantially free of ALDH^{br} cells. The isolated cell populations can be further activated in vitro or ex vivo. This can be accomplished with any monocyte-activating compounds, e.g., LPS, MPLA, or MCP-1.

In another aspect, the invention provides methods for treating ocular vascular disorders. The methods involve administering to a subject suffering from an ocular vascular disorder an isolated monocyte population in an amount that is sufficient to treat or ameliorate the ocular vascular disorder. Preferably, the monocyte population is isolated from a blood sample or a bone marrow sample from the subject. In some preferred embodiments, the subject to be treated with the methods is a human. In some of the methods, the monocyte population comprises substantially pure CD14⁺/CD33⁺ cells. For example, at least 80% of the cells in the isolated monocyte population are CD14⁺/CD33⁺. In some methods, the isolated monocyte population is activated in vitro or ex vivo prior to being administered to the subject. Any compounds known to be able to activate monocytes can be used in these embodiments. For example, the isolated monocyte cells can be activated with LPS, MPLA, or MCP-1. In some methods, an untreated monocyte population (or an in vitro or ex vivo activated monocyte population) is co-administered to a subject along with such a monocyte-activating compound.

Many ocular vascular disorders can be treated with methods of the invention. Examples include ischemic retinopathy, diabetic retinopathy, retinopathy of prematurity, neovascular glaucoma, central retinal vein occlusions, retina edema, macular degeneration and retinitis pigmentosa. In some methods, the isolated monocyte population is administered to the subject via a local route, e.g., via intravitreal injection. In some other methods, the monocyte population is administered to the subject via a systemic route, e.g., via intravenous injection.

In a related aspect, the invention provides other methods of treating or ameliorating an ocular disease in a subject. These methods entail (i) isolating from a blood sample or a bone marrow sample of a subject having an ocular vascular disease a substantially pure monocyte population; and (ii) administering the isolated monocyte population to the subject in an amount sufficient to treat or ameliorate the ocular vascular disease. Some of these methods additionally entail activating the isolated monocyte population ex vivo prior to administering the cells to the subject. Any compounds known to be able to activate monocytes can be used in these embodiments. For example, the isolated monocyte cells can be activated with LPS, MPLA, or MCP-1. In some other embodiments, the isolated monocyte population, with or without further activation ex vivo, is co-administered to the subject along with a monocyte-activating compound.

Typically, the monocyte population used in these methods contains substantially pure CD14⁺/CD33⁺ cells. Preferably, at least about 80% of the cells in the isolated monocyte population express surface markers CD33 and CD14. In some methods, the monocyte population is isolated by (i) debulking red blood cells from the sample; and (ii) separating remaining red blood cells and other cell types in the sample from monocytes based on their size, granularity or density. In some of these methods, the remaining red blood cells and other cell types are separated from monocytes by density centrifugation or fluorescence-activated cell sorting (FACS). Ocular diseases or disorders that are suitable for treatment with these methods include ischemic retinopathy, diabetic retinopathy, retinopathy of prematurity, neovascular glaucoma, central retinal vein occlusions, macular degeneration and retinitis pigmentosa.

In another aspect, the invention provides methods for isolating a substantially pure monocyte population. The methods involve (i) providing a blood sample or a bone marrow sample from a subject; (ii) debulking red blood cells from the sample; and (iii) separating remaining red blood cells and other cell types (platelets, granulocytes and granulocytes) in the sample from monocytes. In some of the methods, the remaining red blood cells and other cell types are separated from monocytes based on their size, granularity or density. In some of the methods, the remaining red blood cells and other cell types are separated from monocytes by density centrifugation or fluorescence-activated cell sorting (FACS). In these methods, the red blood cells can be debulked by Hespan differential centrifugation or Ficoll density gradient centrifugation. These methods can additional include a step of assaying the isolated cell population for expression of surface marker CD14 and CD33.

A further understanding of the nature and advantages of the present invention may be realized by reference to the remaining portions of the specification and claims.

### BRIEF DESCRIPTION OF THE DRAWINGS

FIGS. 1A-1B show properties and therapeutic activities of isolated monocyte populations. (A) Flow cytometry plot showing population of monocytes (gated) that are distinct from lymphocytes. No labeling was used to discriminate these populations; and (B) Data obtained from the mouse oxygen-induced retinopathy model demonstrating that human peripheral blood (HuPB) monocytes isolated in the described manner significantly reduce both neovascular tuft area (black bars) as well as vascular obliteration (white bars) compared to vehicle injection. These results were similar to mouse bone marrow-derived CD44hi cells used as a positive control.

FIGS. 2A-2B show results from flow cytometry analysis of fractions generated by density centrifugation. The data show that the sample is depleted of CD2⁺/CD3⁺ lymphocytes (A) and enriched for CD14⁺/CD33⁺ monocytes (B).

FIG. 3 shows results from ALDH labeling of peripheral blood indicating negligible ALDH^{br}/SSC population.

FIG. 4 shows results from flow cytometry analysis indicating the presence of small number of CD34⁺ cells (top right) relative to the target CD14⁺ monocytes (top left) in the isolated cell population.

FIG. 5 shows post-sort analysis of human peripheral blood monocytes or lymphocytes selected on the basis of light scattering properties as described above. The monocyte fraction is shown to be composed of ˜88% CD14⁺ cells while the lymphocyte population contains virtually no CD14⁺ cells. Also shown is analysis of CD11b and CD33 showing high expression of both of these myeloid markers on the monocyte fraction and few positive cells in the lymphocyte fraction.

FIG. 6 shows results of an in vitro chemotaxis assay showing dose-dependent increase in migration of monocytes (Mono) in response to MCP-1. Lymphocytes (Lympho) failed to respond to MCP-1. Mouse CD44hi bone marrow cells (CD44Hi), which contains monocytes, also responded to MCP-1.

FIG. 7 shows in vitro differential adhesion assay demonstrating the ability of increasing numbers of monocytes to adhere to untreated cell culture plastic. Lymphocytes were unable to adhere in significant number to the same substrate.

FIG. 8 shows images from retinal whole mounts which indicate the presence of GFP-expressing cells in the retina after intracardiac injection 5 days earlier. Injury was created in the retina through exposure to hyperoxia.

FIG. 9 shows cytometric bead array (CBA) analysis of secreted cytokines from LPS-treated monocyte-enriched F5 cells (ActF5). The data showed increased secretion of IL-1 beta, Il-6, IL-8 and TNF after LPS stimulation. For each cytokine, approximate ED50 is given as a reference for quantity and biological activity of protein present in media. Units are in pg/ml.

FIG. 10 shows cytometric bead array data demonstrating increased secretion of cytokines after incubation with LPS, MPLA or mouse MCP-1 for 1 hr or 4 hrs. Two concentrations of LPS and MPLA are shown. Values represent the ratio of the treated (activated) cells to untreated (control) cells.

FIG. 11 shows cytometric bead array data following 4 h and 19 h stimulation with LPS, mouse MCP-1, human MCP-1 and MPLA at different concentrations. The 19 h time point shows that, in addition to LPS and MPLA, mouse and human MCP-1 also stimulate secretion of IL-8 and IL-6, albeit at lower levels.

FIG. 12 shows that activated monocyte-enriched fraction 5 (F5) from both normal and diabetic donors promote vascular repair in the mouse OIR model more effectively than non-activated F5 or other fractions. Data shown are the percentage of retinas within a treatment group with vascular obliteration below 10,000 square microns.

### DETAILED DESCRIPTION OF THE INVENTION

### I. Overview

The present invention relates to isolated and substantially pure populations of monocyte cells which are useful for treating or ameliorating ocular vascular diseases or degenerative disorders. As detailed in the Examples below, the monocyte populations isolated by the present inventors contain substantially pure CD14⁺/CD33⁺ monocytes. The isolated monocyte populations possess the activity of promoting vascular repair as examined in eye disease models. The monocyte populations are also distinct from other known hematopoietic cell populations for clinical use, as evidenced by a lack of AldeFluor Bright labeling and independence on CD34⁺ cells for their therapeutic activities. In addition, some of the isolated cell populations are also characterized by being CD34⁻ and/or containing a very low amount of cells with high level expression of aldehyde dehydrogenase (ALDH^{br} cells). Furthermore, the inventors found that some of the isolated monocyte populations upon activation ex vivo have enhanced ability to promote blood vessel repair. Finally, it was observed that monocytes isolated from donors with retina vascular disorders can also be activated ex vivo and promote vascular repair in a mouse model of ischemic retinopathy, similar to cells isolated from normal donors. These findings provide additional support that therapeutically active monocyte populations can be employed to treat retina vascular disorders in an autologous manner.

The inventors also developed novel procedures for isolating monocyte populations for treating neovascular eye diseases such as macular degeneration and diabetic retinopathy. Utilizing biological samples such as bone marrow, peripheral blood or cord blood, the methods rely on the physical properties of the target cell population and circumvent the need for selection agents such as antibodies that specifically recognize surface antigens of the monocytes. Because of the lack of surface bound heterologous materials such as antibodies, the cell populations isolated with these methods are more desirable for therapeutic uses. A series of in vitro assays were performed to demonstrate the purity and activity of these monocyte preparations. In addition, it was found that monocyte populations isolated using methods disclosed herein possess the desired therapeutic activity in a model of ischemic retinopathy.

In accordance with these discoveries, the present invention provides isolated or substantially purified monocyte populations that are therapeutically effective. The invention also provides novel methods for isolating such monocyte populations. The invention further provides methods of treating or ameliorating diseases or disorders related to or mediated by aberrant ocular vascularization. Additionally, methods are provided for producing highly active monocyte cells by in vitro or ex vivo activation with compounds capable of activating monocyte (e.g., agonist compounds of CD14 or TLR4), as well as methods for identifying novel compounds that can activate monocyte cells in a similar fashion. The invention also encompasses therapeutic methods using a combination of the isolated monocyte populations and a compound capable of activating and recruiting the cells (e.g., MCP-1). In these methods, the cells can be activated upon administration to the subject, and a sustained effect can be mediated by additional recruited cells.

The highly activated cells and the novel activating compounds are useful in the treatment of various eye diseases. Examples of such diseases include diabetic retinopathy, diabetic macular edema, retinal vein occlusions, retinopathy of prematurity, age-related macular degeneration, retinal angiomatous proliferation, macular telangectasia, ischemic retinopathy, iris neovascularization, intraocular neovascularization, corneal neovascularization, retinal neovascularization, choroidal neovascularization, and retinal degeneration. Subjects suitable for treatment with methods of the invention include ones who have or are at risk of developing any of these diseases. The following sections provide more detailed guidance for practicing the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Illustrated Dictionary of Immunology, Cruse (Ed.), CRC Pr I Llc (2^{nd} ed., 2002); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

Hematopoietic stem cells are stem cells that are capable of developing into various blood cell types e.g., B cells, T cells, granulocytes, platelets, and erythrocytes. The lineage surface antigens (surface markers) are a group of cell-surface proteins that are markers of mature blood cell lineages, including CD2, CD3, CD11, CD11a, Mac-1 (CD11b:CD18), CD14, CD16, CD19, CD24, CD33, CD36, CD38, CD45, CD45RA, murine Ly-6G, murine TER-119, CD56, CD64, CD68, CD86 (B7.2), CD66b, human leukocyte antigen DR (HLA-DR), and CD235a (Glycophorin A). Hematopoietic stem cells that do not express significant levels of these antigens are commonly referred to a lineage negative (Lin₋). Human hematopoietic stem cells commonly express other surface antigens such as CD31, CD34, CD117 (c-kit) and/or CD133. Murine hematopoietic stem cells commonly express other surface antigens such as CD34, CD117 (c-kit), Thy-1, and/or Sca-1.

The cells that circulate in the bloodstream are generally divided into three types: white blood cells (leukocytes), red blood cells (erythrocytes), and platelets or thrombocytes. Leukocytes include granulocytes (polymorphonuclear leukocytes) and agranulocytes (mononuclear leucocytes). Granulocytes are leukocytes characterized by the presence of differently staining granules in their cytoplasm when viewed under light microscopy. There are three types of granulocytes: neutrophils, basophils, and eosinophils. Agranulocytes (mononuclear leucocytes) are leukocytes characterized by the apparent absence of granules in their cytoplasm. Although the name implies a lack of granules, these cells do contain non-specific azurophilic granules, which are lysosomes. Agranulocytes include lymphocytes, monocytes, and macrophages.

Monocytes are produced by the bone marrow from haematopoietic stem cell precursors called monoblasts. Monocytes circulate in the bloodstream for about one to three days and then typically move into tissues throughout the body. They constitute between three to eight percent of the leukocytes in the blood. In the tissues monocytes mature into different types of macrophages at different anatomical locations. Monocytes have two main functions in the immune system: (1) replenish resident macrophages and dendritic cells under normal states, and (2) in response to inflammation signals, monocytes can move quickly (aprox. 8-12 hours) to sites of infection in the tissues and divide/differentiate into macrophages and dendritic cells to elicit an immune response. Monocytes are usually identified in stained smears by their large bilobate nucleus.

Ocular neovascularization or ocular vascular disorder is a pathological condition characterized by altered or unregulated proliferation and invasion of new blood vessels into the structures of ocular tissues such as the retina or cornea. Examples of ocular neovascular diseases include ischemic retinopathy, iris neovascularization, intraocular neovascularization, age-related macular degeneration, corneal neovascularization, retinal neovascularization, choroidal neovascularization, diabetic retinal ischemia, retinal degeneration and diabetic retinopathy.

Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, retinitis pigmentosa, retina edema (including macular edema), Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

Age-related macular degeneration begins with characteristic yellow deposits in the macula (central area of the retina which provides detailed central vision, called fovea) called drusen between the retinal pigment epithelium and the underlying choroid. Most people with these early changes (referred to as age-related maculopathy) have good vision. People with drusen can go on to develop advanced AMD. The risk is considerably higher when the drusen are large and numerous and associated with disturbance in the pigmented cell layer under the macula. Large and soft drusen are related to elevated cholesterol deposits and may respond to cholesterol lowering agents or the Rheo Procedure.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, a yellow central area of the retina, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

The terms “subject” and “patient” are used interchangeably and refer to mammals such as human patients and non-human primates, as well as experimental animals such as rabbits, rats, and mice, and other animals. Animals include all vertebrates, e.g., mammals and non-mammals, such as dogs, cats, sheeps, cows, pigs, rabbits, chickens, and etc. Preferred subjects for practicing the therapeutic methods of the present invention are human. Subjects in need of treatment include patients already suffering from an ocular vascular disease or disorder as well as those prone to developing the disorder.

The term “substantially pure” or “substantial purity” when referring to an isolated cell population means the percentage of a given cell (target cell) in the population is significantly higher than that found in a natural environment (e.g., in a tissue or a blood stream of a subject). Typically, percentage of the target cell (e.g., monocyte) in a substantially pure cell population is at least about 50%, preferably at least about 60%, 70%, 75%, and more preferably at least about 80%, 85%, 90% or 95% of total cells in the cell population.

As used herein, “treating” or “ameliorating” includes (i) preventing a pathologic condition (e.g., macular degeneration) from occurring (e.g. prophylaxis); (ii) inhibiting the pathologic condition (e.g., macular degeneration) or arresting its development; and (iii) relieving symptoms associated with the pathologic condition (e.g., macular degeneration). Thus, “treatment” includes the administration of an isolated cell population of the invention and/or other therapeutic compositions or agents to prevent or delay the onset of the symptoms, complications, or biochemical indicia of an ocular disease described herein, alleviating or ameliorating the symptoms or arresting or inhibiting further development of the disease, condition, or disorder. “Treatment” further refers to any indicia of success in the treatment or amelioration or prevention of the ocular disease, condition, or disorder described herein, including any objective or subjective parameter such as abatement; remission; diminishing of symptoms or making the disease condition more tolerable to the patient; slowing in the rate of degeneration or decline; or making the final point of degeneration less debilitating. Detailed procedures for the treatment or amelioration of an ocular disorder or symptoms thereof can be based on objective or subjective parameters, including the results of an examination by a physician.

### III. Methods of Isolating Population of Monocyte Cells

The invention provides methods for isolating a population of monocytes that are useful to treat various ocular vascular disorders as described herein. As exemplified in the Examples below, the monocyte populations can be isolated from suitable biological samples obtained from a mammalian subject, e.g., peripheral blood or bone marrow. The methods of the present invention enable isolation of substantially pure (e.g., with at least 50%, 75% or 85% purity) monocyte populations from a bone marrow or a blood sample. The blood sample can be any sample that contains the bulk of white blood cells or mononuclear leukocytes from whole blood. For example, it can be whole blood or leukapheresis product from whole blood. Leukapheresis is a laboratory procedure in which white blood cells are separated from a sample of blood. Preferably, the monocytes present in the isolated cell populations are CD14⁺/CD33⁺. CD33 is a transmembrane receptor expressed on cells of monocytic/myeloid lineage. CD14 is a membrane-associated glycosylphosphatidylinositol-linked protein expressed at the surface of cells, especially macrophages. Bone marrow, peripheral blood, and umbilical cord blood each include a sub-population of monocytes that express the CD14 antigen and CD33. Thus, these biological samples are preferred for isolating monocyte populations enriched for CD14⁺ and CD33⁺ cells in accordance with the methods disclosed herein. In some embodiments, the isolated cell populations are also characterized by being CD34- and/or expressing no or low levels of aldehyde dehydrogenase (ALDH). Preferably, the monocyte populations are isolated from human bone marrow, human peripheral blood, human umbilical cord blood or other related blood samples.

Typically, the methods entail first removal the majority of red blood cells (RBCs) from the sample (“debulking”). This step is accompanied by separation of other blood cells (e.g., platelets, granulocytes and lymphocytes) and remaining red blood cells, if any, from monocytes. Unlike methods known in the art, no labeling agents (e.g., antibodies) which recognize cell surface markers of the different cell types are used in the methods of the present invention. Instead, the present invention separate monocytes from other blood cell types, especially other mononuclear cells (e.g. lymphocytes) based only on physical properties such as size, granularity and density. In some embodiments, monocyte populations of the present invention are isolated from a suitable sample such as bone marrow or peripheral blood via a method based on fluorescence-activated cell sorting (FACS). As detailed in the Examples below, RBCs present in a biological sample (e.g., peripheral blood) from a mammalian subject are first removed in the isolation procedures. This can be accomplished by lysing RBCs with standard procedures well known in the art, e.g., ammonium chloride-based lysing method. See, e.g., Tiirikainen, Cytometry 20:341-8, 1995; and Simon et al., Immunol. Commun. 12:301-14, 1983. Alternatively, RBCs can be sedimented and mononuclear cells separated by centrifugation on ficoll. Procedures for separating red blood cells via ficoll density gradient centrifugation are described in the art, e.g., Tripodi et al., Transplantation. 11:487-8, 1971; Vissers et al., J. Immunol. Methods. 110:203-7, 1988; and Boyum et al., Scand. J. Immunol. 34:697-712, 1991. Another method suitable for debulking RBCs is by differential centrifugation using the ability of Hespan (Dupont, Dreieich, Germany) to induce red blood cell agglutination. See, e.g., Nagler et al., Exp. Hematol. 22:1134-40, 1994; and Pick et al., Br. J. Haematol. 103:639-50, 1998. Further techniques that can be used to debulk RBCs include the use of blood cell filters. Such blood cell filters are readily available from commercial suppliers, e.g., the leukocyte depleting filter manufactured by Pall Biomedical Products Company (East Hills, N.Y.).

After the removal of RBCs, remaining cells in the sample are suspended in an appropriate buffer that is suitable for the subsequent isolation step with FACS. For example, the cells can be resuspended in DPBS/0.5% BSA/2 mM EDTA. Flow cytometry is a technique for counting, examining, and sorting microscopic particles suspended in a stream of fluid. It allows simultaneous multiparametric analysis of the physical and/or chemical characteristics of single cells flowing through an optical and/or electronic detection apparatus. Typically, a beam of light (usually laser light) of a single wavelength is directed onto a hydro-dynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals found in the particle or attached to the particle may be excited into emitting light at a lower frequency than the light source. This combination of scattered and fluorescent light is picked up by the detectors, and by analyzing fluctuations in brightness at each detector (one for each fluorescent emission peak) it is then possible to derive various types of information about the physical and chemical structure of each individual particle. FSC correlates with the cell volume and SSC depends on the inner complexity of the particle (i.e. shape of the nucleus, the amount and type of cytoplasmic granules or the membrane roughness). Some flow cytometers on the market have eliminated the need for fluorescence and use only light scatter for measurement. Other flow cytometers form images of each cell's fluorescence, scattered light, and transmitted light.

Modern flow cytometers are able to analyze several thousand particles every second in real time, and can actively separate and isolate particles having specified properties. A flow cytometer is similar to a microscope, except that instead of producing an image of the cell, flow cytometry offers high-throughput automated quantification of set parameters. A flow cytometer has 5 main components: a flow cell-liquid stream, a light source (e.g., laser), a detector and Analogue to Digital Conversion (ADC) system which generate FSC and SSC as well as fluorescence signals, an amplification system, and a computer for analysis of the signals. The data generated by flow-cytometers can be plotted in a single dimension, to produce a histogram, or in two dimensional dot plots or even in three dimensions. The regions on these plots can be sequentially separated, based on fluorescence intensity, by creating a series of subset extractions, termed “gates”. Specific gating protocols exist for diagnostic and clinical purposes especially in relation to haematology. The plots are often made on logarithmic scales. Because different fluorescent dyes' emission spectra overlap, signals at the detectors have to be compensated electronically as well as computationally.

Fluorescence-activated cell sorting (FACS) is a specialized type of flow cytometry. It provides a method for sorting a heterogeneous mixture of biological cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest. The cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge. In some systems the charge is applied directly to the stream and the droplet breaking off retains charge of the same sign as the stream. The stream is then returned to neutral after the droplet breaks off.

As an example of the present invention, FACS can be carried out on a BD FACSAria Cell-Sorting System (BD Biosciences, San Jose, Calif.) using a series of gates. No antibodies or other selection agents are used in the sorting. Dead cells and debris can be first gated out by drawing a region that includes only viable white blood cells. Thereafter, doublets or aggregated cells can be removed with secondary and tertiary gates that interrogate forward scatter width (FSC-W) vs. forward scatter area (FSC-A) and side scatter width (SSC-W) vs. side scatter area (SSC-A), respectively. The procedures can be performed in accordance with standard protocols well known in the art, e.g., Flow cytometry—A practical approach, Ormerod (ed.), Oxford University Press, Oxford, UK (3^{rd} ed., 2000); and Handbook of Flow Cytometry Methods, Robinson et al. (eds.), Wiley-Liss, New York (1993).

In some other embodiments, monocyte populations of the invention are isolated using a separation scheme based on the Elutra® Cell Separation System (Gambro BCT Inc., Lakewood, Colo.). Elutra® is a semi-automatic, centrifuge-based instrument using continuous counter-flow elutriation technology to separate cells into multiple fractions based on size and density. Prior to separation with Elutra®, the biological sample obtained from a mammalian subject (e.g., a peripheral blood sample from a human patient) can be first treated to remove the bulk of RBCs, e.g., by sedimentation with HESpan. Nucleated cell fraction can then be collected, e.g., with a plasma expressor, before being processed with the Elutra® device. As detailed in the Examples below, fractionation by the Elutra® device allows separation of monocytes from platelets, remaining RBCs, lymphocytes and granulocytes. The fractionated cells can be further analyzed for cell count, viability and purity.

In some embodiments, the invention provides methods for producing highly active cells for therapeutic applications. In these embodiments, monocyte populations isolated in accordance with the present disclosure are further activated in vitro or ex vivo prior to being administered to a subject with ocular vascular disorders. Typically, the cells are treated with a compound that is capable of activating monocytes. Detailed procedures for activating isolated monocyte populations are described below.

### IV. Properties and Activities of Isolated Monocyte Populations

Monocyte populations isolated from biological samples such as whole blood or bone marrow can be examined for their immunological or biological properties, as well as their therapeutic activities. As detailed in the Examples, purity and activities of the isolated monocyte populations can be assessed with a number of assays. For example, to analyze surface marker expressions, some methods of the invention can further involve a step of assessing expression of CD14 and CD33 by the isolated monocyte populations. Surface marker expressions of the isolated cells can be examined with anti-CD14 and anti-CD33 monoclonal antibodies in conjunction with flow cytometry. As exemplified in the Examples below, cell populations isolated with methods of the present invention contain substantially purified CD14⁺/CD33⁺ monocytes. For example, the isolated cell populations can have at least 50%, 60%, 75%, 80%, 85%, 90% or 95% of cells expressing CD14 and CD33.

In addition to their substantial purity, the isolated cell populations are functionally effective to treat or ameliorate symptoms associated with ocular vascular disorders. For example, as disclosed herein, the isolated cell populations can promote vascular repair in oxygen-induced retinopathy in mice. Mouse model of ischemic retinopathy and its use in assessing therapeutic activities of isolated cell populations for ocular vascularization disorders are described in the art. See, e.g., Ritter et al., J. Clin. Invest. 116:3266-76, 2006; and Ritter et al., Invest. Ophthalmol. Vis. Sci. 46:3021-6, 2005.

Function and biochemical activity of the isolated cells can also be analyzed by measuring chemotaxis of the cells, e.g., using a monocyte chemotactic protein such as MCP-1. Results from such an activity assay also provide a readout of the relative purity of the preparation and an indication of the viability and function of the isolated cells. Additional methods for examining purity and viability of the isolated monocytes include an assay that is based on differential adhesion to cell culture substrata by monocytes relative to other monoclear cells. As demonstrated in the Examples, it was found that cells generated by the isolation methods of the invention are primarily monocytes as evidenced by their ability to adhere under the described assay conditions.

Some of the isolated monocyte populations of the invention are also CD34⁻. The CD34⁻ monocyte populations of the invention are defined as monocyte populations that, in addition to being CD14⁺ and CD33⁺, contain no or very low levels (e.g., less than about 5%, 4%, 3%, 2%, 1%, 0.5%, 0.25%, 0.1%, 0.05% or 0.01%) of CD34⁺ cells. The presence of CD34⁺ cells in a cell population can be readily determined and quantified using methods well known in the art or disclosed herein. The CD34⁻ monocyte populations of the invention are more suitable for use in some therapeutic applications of the present invention. CD34⁺ stem cells are known to have the potential to differentiate into unwanted cell types and may have proliferative capacity. Such properties of CD34⁺ cells can be undesirable in the practice of the presently disclosed therapeutic methods. It was found that injection of undifferentiated stem cell populations, such as CD34⁺ stem cells, into the mouse eye resulted in a poor outcome (Example 3). Thus, in addition to being CD14⁺/CD33⁺, some of the monocyte populations of the present invention are also characterized by a lack of CD34⁺ cells or a very low amount of CD34⁺ cells. As exemplified in the Examples below, a small amount of CD34⁺ cells that may be present in the initial cell preparations can be further depleted from the final isolated monocyte populations. Importantly, as disclosed herein, removal of the CD34⁺ cells does not result in any change of the therapeutic activities of the monocyte populations.

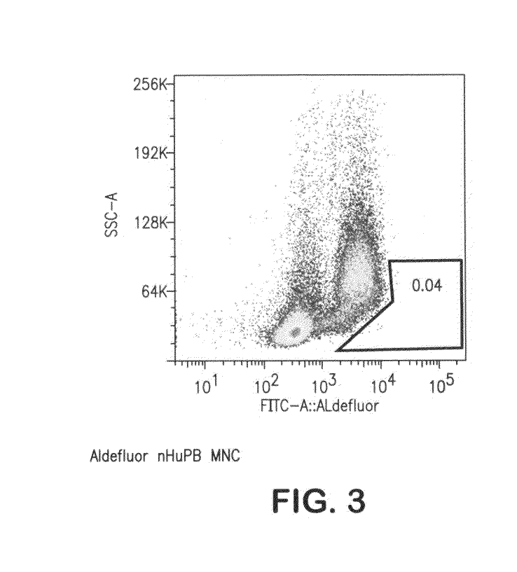

In some other embodiments, the monocyte populations of the invention are also characterized by containing no, or being substantially free of, cells with high expression of aldehyde dehydrogenase (ALDH^{br} cells). ALDH^{br} cells are well known in the art. They have progenitor cell activity and have been suggested to be useful in cell therapy applications (Gentry et al., Cytother. 9:259-274, 2007). Presence of ALDH^{br} cells in a cell population can be typically sorted and quantified via fluorescence-activated cell sorting (FACS) as described in the Examples herein and also in the art, e.g., Russo et al., Biochem. Pharmacol. 37:1639-1642, 1988; and Storms et al., Blood 106:95-102, 2005. As shown in FIG. 3, some of the isolated monocyte populations of the invention contain negligible amount (about 0.04%) of ALDH^{br} cells. Thus, some preferred embodiments of the invention provide isolated or purified monocyte populations that are substantially free of ALDH^{br} cells. As measured by fluorescence-activated cell sorting, these monocyte cell populations should contain less than about 5%, 2%, or 1% of ALDH^{br} cells. More preferably, the percentage of ALDH^{br} cells in these cell populations should be less than 0.5%, less than 0.1%, or less than 0.05%. By being both CD34⁻ and/or ALDH low, these CD14⁺/CD33⁺ monocyte populations of the invention are further distinguished from other blood cell or stem cell populations that have been reported in the art (see, e.g., Storms et al., Blood 106:95-102, 2005).

Cells from the monocyte populations of the present invention can also be engineered to express a therapeutically useful agent, such as antiangiogenic agents for use in cell-based gene therapy or neurotrophic agents to enhance neuronal rescue effects. In these embodiments, the isolated monocyte cell populations are transfected with a gene that encodes the therapeutically useful agent. Suitable genes and methods for transfection into cells of the monocyte populations of the present invention are described in, e.g., U.S. patent application Ser. No. 10/080,839. In some of these embodiments, the cells are transfected with a polynucleotide that operably encodes an angiogenesis inhibiting peptide, e.g., TrpRS or antiangiogenic (i.e., angiostatic) fragments thereof (see, e.g., U.S. patent application Ser. No. 11/884,958). The engineered angiogenesis inhibiting cells from the monocyte cell population are useful for modulating abnormal blood vessel growth in diseases associated with abnormal vascular development, such as ARMD, diabetic retinopathy, and certain retinal degenerations and like diseases. In some other embodiments, cells of the isolated monocyte cell population of the present invention are transfected to express a gene encoding a neurotrophic agent. The neurotrophic agent expressed by the transfected gene can be, e.g., nerve growth factor, neurotrophin-3, neurotrophin-4, neurotrophin-5, ciliary neurotrophic factor, retinal pigmented epithelium-derived neurotrophic factor, insulin-like growth factor, glial cell line-derived neurotrophic factor, brain-derived neurotrophic factor, and the like. The monocyte cells transfected with such a gene are useful for promoting neuronal rescue in ocular diseases involving retinal neural degeneration, such as glaucoma, retinitis pigmentosa, injuries to the retinal nerves, and the like. See, e.g., Kirby et al., Mol. Ther. 3:241-8, 2001; Farrar et al., EMBO J. 21:857-864, 2002; Fournier et al., J. Neurosci. Res. 47:561-572, 1997; and McGee et al., Mol. Ther. 4:622-9, 2001.

### V. Treating Ocular Vascular Diseases

The present invention provides methods of treating or ameliorating vascular disorders and neuronal degeneration in the retina of a mammal that suffers from an ocular disease. In accordance with the methods, isolated monocyte populations or engineered cells thereof as described above can be administered to the retina of the mammal, either by intravitreal injection or systemic administration. The cells are administered in an amount sufficient to ameliorate vascular and/or neuronal degeneration in the retina. Preferably, the isolated monocyte population is autologous to the mammal to be treated. Preferably, the isolated monocyte cells are administered in a physiologically tolerable medium, such as phosphate buffered saline (PBS).

In some of the therapeutic methods, a monocyte population containing substantially purified (e.g., at least 75% or 80%) CD14⁺/CD33⁺ cells is first isolated from a whole blood sample or a bone marrow sample obtained from the subject to be treated. The monocyte cell population is isolated using the methods described above. The isolated CD14⁺/CD33⁺ monocyte population is then administered to the subject in an amount that is sufficient to ameliorate or treat the vascular and/or neuronal degeneration of the retina. The cells can be isolated from a mammal suffering from an ocular degenerative disease or ocular vascular disease, preferably at an early stage of the ocular disease or from a healthy subject known to be predisposed to the development of an ocular degenerative disease (i.e., through genetic predisposition). In the latter case, the isolated monocyte population can be stored after isolation, and can then be injected prophylactically during early stages of a later developed ocular disease.

Not intended to be bound in theory, cells from the CD14⁺/CD33⁺ monocyte population of the invention may exert their therapeutic effect by selectively targeting astrocytes, incorporating into developing vasculature and then differentiating to become vascular endothelial cells. The cells may promote neuronal rescue in the retina and promote upregulation of anti-apoptotic genes. When systemically administered or intravitreally injected into the eye of a mammalian subject (e.g., a human or a mouse) from which the cells were isolated, the cells are useful for the treatment of retinal neovascular and retinal vascular degenerative diseases, and for repair of retinal vascular injury.

The subjects suitable for treatment with methods of the invention can be neonatal, juvenile or fully mature adults. In some embodiments, the subjects to be treated are neonatal subjects suffering from ocular disorders such as oxygen induced retinopathy or retinopathy of prematurity. In some embodiments, the subjects are human, and the isolated monocyte populations to be used are human cells, preferably autologous cells isolated from the subject to be treated. Subjects suffering from various ocular vascular diseases or ocular degenerative disorders are suitable for treatment with the monocyte populations of the invention. These include ocular diseases such as retinal degenerative diseases, retinal vascular degenerative diseases, retina edema (including macular edema), ischemic retinopathies, vascular hemorrhages, vascular leakage, choroidopathies, retinal injuries and retinal defects involving an interruption in or degradation of the retinal vasculature. Specific examples of such diseases include age related macular degeneration (ARMD), diabetic retinopathy (DR), presumed ocular histoplasmosis (POHS), retinopathy of prematurity (ROP), sickle cell anemia, and retinitis pigmentosa, as well as retinal injuries. In addition, the monocyte populations also can be used to generate a line of genetically identical cells, i.e., clones, for use in regenerative or reparative treatment of retinal vasculature, as well as for treatment or amelioration of retinal neuronal degeneration. Further more, the monocyte populations of the invention are useful as research tools to study retinal vascular development and to deliver genes to selected cell targets, such as astrocytes.

For therapeutic or prophylactic applications, the isolated monocyte population of the invention can be administered to the subject via either a local route or a systemic route. In some embodiments, local administration of the cells is desired in order to achieve the intended therapeutic effect. For example, the cell population can be administered to the subject by intraocular injection (intravitreal injection). This can be performed in accordance with standard procedures known in the art. See, e.g., Ritter et al., J. Clin. Invest. 116:3266-76, 2006; Russelakis-Carneiro et al., Neuropathol. Appl. Neurobiol. 25:196-206, 1999; and Wray et al., Arch. Neurol. 33:183-5, 1976. In some other therapeutic methods of the invention, a systemic route of administration of the isolated monocyte population is employed. For example, the cells can be administered to the subject by intravenous injection that is routinely practiced in the art. In some other embodiments, non-human subjects may also be administered with the cells via intracardiac injection. This can be accomplished based on procedures routinely practiced in the art. See, e.g., Iwasaki et al., Jpn. J. Cancer Res. 88:861-6, 1997; Jespersen et al., Eur. Heart J. 11:269-74, 1990; and Martens, Resuscitation 27:177, 1994. Other routes of administration may also be employed in the practice of the present invention. See, e.g., Remington: The Science and Practice of Pharmacy, Mack Publishing Co., 20^{th} ed., 2000.

In general, the number of cells from the monocyte population injected into the eye should be sufficient for arresting the disease state of the eye. For example, the amount of injected cells can be effective for repairing retinal damage of the eye, stabilizing retinal neovasculature, maturing retinal neovasculature, and preventing or repairing vascular leakage and vascular hemorrhage. Typically, for intravitreal injection, at least about 1×10⁴, at least 1×10⁵, or at least 1×10⁶ cells from the isolated monocyte population or transfected cells from the monocyte population are injected to an eye of the subject suffering from an ocular vascular disorder (e.g., a retinal degenerative disease). The number of cells to be injected may depend upon the severity of the retinal degeneration, the age of the subject and other factors that will be readily apparent to one of ordinary skill in the art of treating ocular diseases. The cells from the monocyte population may be administered in a single dose or by multiple dose administration over a period of time, as may be determined by the physician in charge of the treatment. Also, the number of cells and frequency of administration can vary depending on whether the treatment is prophylactic or therapeutic. In prophylactic applications, a relatively low number of cells may be administered at relatively infrequent intervals over a long period of time. Some subjects may continue to receive treatment for the rest of their lives. In therapeutic applications, a relatively high number of cells at relatively short intervals may be required until progression of the disease is reduced or terminated, and preferably until the subject shows partial or complete amelioration of symptoms of the ocular vascular disease. Thereafter, the subject can be administered a prophylactic regime.

### VI. Enhancing Activities of Isolated Monocyte Populations Via Ex Vivo Activation

In the various therapeutic applications described above, the isolated monocyte populations or engineered cells thereof can also be activated in vitro or ex vivo prior to being administered to a subject in need of treatment. In these embodiments, enhanced therapeutic activities can be achieved when the ex vivo activated monocytes are administered to the retina of subjects afflicted with ocular vascular disorders.

Activation of the isolated monocyte populations can be readily carried out in accordance with materials and methods routinely practiced in the art or exemplified in the Examples below. Monocytes and macrophages are known to be activated by a variety of agents such as LPS, through CD14 and toll-like receptors (Le-Barillec et al., J. Leukoc. Biol. 68:209-15, 2000; Mirlashari et al., Med. Sci. Monit. 9:BR316-24, 2003). As demonstrated in the Examples below, the isolated monocyte populations can be activated with diverse agents such as lipopolysaccharide (LPS), monophosphoryl lipid A (MPLA) and monocyte chemotactic protein 1 (MCP-1). It was also shown that, relative to untreated cells, the activated cells produced better therapeutic results in animals with oxygen-induced retinopathy. Thus, these agents can be readily employed for ex vivo activation of the isolated monocyte populations. Many other monocyte-activating compounds that are known in the art can also be used in the practice of the present invention. Examples of such compounds include immunomodulators (such as gamma interferon, lymphokines, muramyl dipeptide), phorbol myristate acetate, concanavalin A, polymethylmethacrylate, and dietary fats. See, e.g., Koff et al., Science 224:1007-1009, 1984; Chung et al., J. Leukoc. Biol. 44:329-336, 1988; Horwitz et al., J. Exp. Med. 154:1618-1635, 1981; Laing et al., Acta Orthop. 79:134-40, 2008; Bently et al., Biochem. Soc. Trans. 35:464-5, 2007.

To activate the monocytes, the isolated cells can be incubated with any one of the compounds at an appropriate concentration for a sufficient period of time. The amount of compounds to be used and the length of the time for the activation prior to administration of the cells can be determined empirically or in accordance with teachings of the art. Specific guidance for activating isolated monocyte populations with some of the compounds is also provided in the Examples below. Using LPS as an example, the cells can be incubated with LPS at a concentration of about 1 ng/ml to about 1000 ng/ml, preferably at a concentration of about 5 ng/ml to about 200 ng/ml or from about 20 ng/ml to about 50 ng/ml. The cells are typically treated with an activating compound for at least 10 minutes, preferably at least an hour prior to being used in therapeutic applications. In some embodiments, the cells are treated with the compound for at least 2 hours, at least 4 hours, at least 10 hours, at least 24 hours or longer.

Prior to administering the treated cells to a subject, the cells can also be examined in vitro to ascertain their activation. This can be typically carried out by qualitatively or quantitatively monitoring cytokine secretions by the treated monocytes. As shown in the Examples, activated monocytes have increased secretions of cytokines such as IL-1β, IL-8, IL-6 and TNF. As exemplified in the Examples, cytokine secretion profiles of monocytes can be easily assessed with routinely practiced methods such as cytometric bead array (CBD) analysis. See e.g., Elshal et al., Methods. 38:317-329, 2006; and Morgan et al., Clin. Immunol. 110:252-266, 2004.

Other than activating an isolated monocyte population in vitro or ex vivo before administering the cells to a subject, some therapeutic methods of the invention involve co-administering to the subject an untreated monocyte population and a monocyte-activating compound disclosed herein (e.g., MCP-1). In some related embodiments, the subject in need of treatment is administered with an in vitro or ex vivo activated monocyte population along with a monocyte-activating compound described above (e.g., MCP-1). In these embodiments, the co-administered compound can activate the administered monocytes in vivo or reinforce activities of the treated cells in vivo.

In a related aspect, the invention provides methods for identifying novel compounds that are capable of activating and stimulating therapeutic activities of monocytes. Typically, these methods entail contacting a candidate compound with a population of monocytes or macrophage (e.g., a monocyte population described herein) and monitoring a parameter of the monocytes that is indicative of an activated status of the cell population. The parameter to be monitored can be any biological, biochemical or morphological characteristics of the cells. In some preferred embodiments, the cells treated with a candidate agent are examined for secretion levels of one or more cytokines such as IL-6, IL8 or TNF. An increased secretion of one or more of these cytokines by the treated cells relative to untreated cells indicates that the candidate compound is a novel monocyte-activating compound.

Candidate compounds to be screened in the methods can be from of chemical classes, including small organic molecules, proteins, polypeptides, polysaccharides, polynucleotides, and the like. In some preferred embodiments, the candidate compounds are small molecule organic agents (e.g., organic compounds of less than about 500 daltons or less than about 1,000 daltons). Preferably, high throughput assays are adapted and employed to screen combinatorial libraries of candidate compounds (e.g., libraries of small organic molecules). Such assays are well known in the art, e.g., as described in Schultz (1998) Bioorg Med Chem Lett 8:2409-2414; Weller (1997) Mol Divers. 3:61-70; Fernandes (1998) Curr. Opin. Chem. Biol. 2:597-603; and Sittampalam (1997) Curr. Opin. Chem. Biol. 1:384-91. Large combinatorial libraries of candidate compounds can be constructed by the encoded synthetic libraries (ESL) method described in WO 95/12608, WO 93/06121, WO 94/08051, WO 95/35503 and WO 95/30642. Other methods for synthesizing various libraries of compounds are described in, e.g., by Overman, Organic Reactions, Volumes 1-62, Wiley-Interscience (2003); Broom et al., Fed Proc. 45: 2779-83, 1986; Ben-Menahem et al., Recent Prog. Horm. Res. 54:271-88, 1999; Schramm et al., Annu. Rev. Biochem. 67: 693-720, 1998; Bolin et al., Biopolymers 37: 57-66, 1995; Karten et al., Endocr. Rev. 7: 44-66, 1986; Ho et al., Tactics of Organic Synthesis, Wiley-Interscience; (1994); and Scheit et al., Nucleotide Analogs: Synthesis and Biological Function, John Wiley & Sons (1980).

### EXAMPLES

The following examples are provided to further illustrate the invention but not to limit its scope. Other variants of the invention will be readily apparent to one of ordinary skill in the art and are encompassed by the appended claims.

### Example 1

### Isolating Monocyte Populations

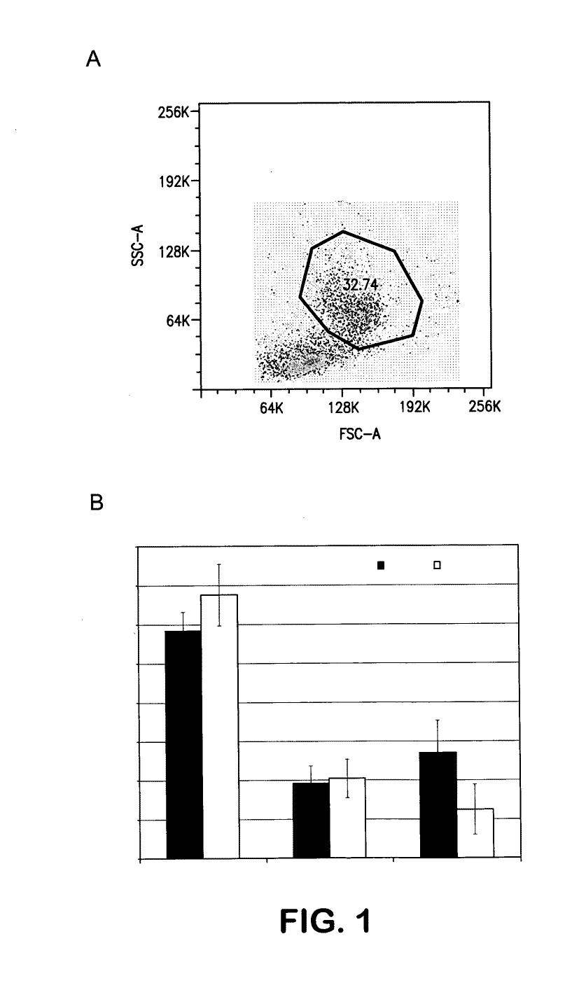

Peripheral blood or bone marrow can be used as a source material for the procedures described here. As an example, we have selected peripheral blood as a cell source due to the relative abundance of monocytes and the ease/safety of collection versus bone marrow. For therapeutic use it is desirable to have cells that are free of any bound compounds related to selection. With this goal in mind, we have conceived and put into practice methods that distinguish monocytes from other mononuclear cells (e.g. lymphocytes) based only on physical properties such as size, granularity and density. The first method we have developed is based on FACS for sensitively separating monocytes from lymphocytes based on differences in cell size and granularity, without the use of antibodies. Results showing monocyte populations isolated with this method is indicated in FIG. 1A. Prior to FACS based separation, the erythrocytes and granulocytes present in whole blood are removed during a pre-sort Ficoll centrifugation step. This can be achieved with several means, e.g., (1) ammonium chloride can be used to lyse RBCs, (2) RBCs can be sedimented and mononuclear cells isolated by centrifugation on ficoll, and (3) RBCs can also be sedimented using Hespan.

Following RBC debulking, cells are suspended in DPBS/0.5% BSA/2 mM EDTA in preparation for fluorescence-activated cell sorting (FACS). Sorting is carried out on a BD Biosciences ARIA using a series of gates and no antibody or other selection agent. Dead cells and debris are first gated out by drawing a region that includes only viable white blood cells. Next, doublets or aggregated cells are removed with secondary and tertiary gates that interrogate forward scatter width (FSC-W) vs. forward scatter area (FSC-A) and side scatter width (SSC-W) vs. side scatter area (SSC-A), respectively. With only single white blood cells under consideration, a gate is drawn in FSC-A vs. SSC-A mode to select cells that are found in a region that reproducibly contains monocytes. Using the assays described in Example 2, we found that cell populations obtained with this method contain CD14⁺/CD33⁺ monocytes with purities of 80%-85%.

A second method of isolating monocyte populations which discriminates cells based on density relies on differential mobility during centrifugation. This method has certain advantages in clinical applications because disposable tubing sets can be used to ensure sterility and eliminate cross-contamination of samples. Specifically, human blood sample was first treated to debulk red blood cells (RBCs) by sedimentation using HESpan. Thereafter, an appropriate volume of 6% HESpan was added to anti-coagulated blood product to reach final concentration of 1.5%. The bag was gently mixed and was incubated upright, at room temperature for 45 minutes to allow the RBCs to sediment. The nucleated cell fraction (NCF) was then expressed off using a manual plasma expressor and collected into a separate sterile 600 mL empty blood bag. The resulting cell product was used as the starting material for further separation based on gradient density centrifugation.

An Elutra® device (Gambro BCT Inc., Lakewood, Colo.) designed to enrich for Monocyte population was then utilized for processing the starting cell product. The disposable tubing set was connected to the Elutra® device. The starting cell product, primary and secondary media bags containing HBSS and 0.5% HSA were then connected to the appropriate connection on the tubing set. The tubing set was primed using the secondary bag. The program number one (see table 1 below) was used to process the starting cell product. The program automatically loaded the starting cell product into the chamber and processed it using the primary media bag. The cells were then continuously centrifuged, separated and collected in multiple fractions at various flow rates. The program was designed to collect 5 fractions each enriched with a particular cell population as follows. Platelets were collected in fraction one, RBC in fraction two, lymphocytes in fraction three, monocytes in fraction four and granulocytes in fraction five. Each fraction was sampled and analyzed for cell count, viability by nuclear cell counter and purity by flow cytometry. The flow rates and collection volumes for each fraction are shown in Table 1. Based on the purity and cell count, appropriate volume containing monocytes was collected and then centrifuged at 300×g.

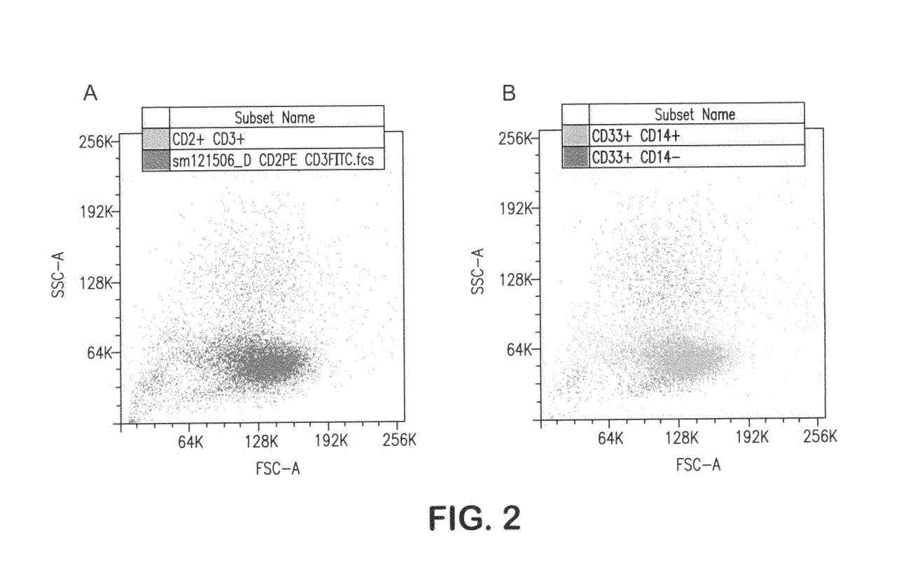

As indicated in FIG. 2, monocyte preparations isolated by the density centrifugation method were found to be similar in nature to those separated by the FACS-based method.

TABLE 1

| Fraction | Flow Rate | Centrifugation Speed | Collection Volume |
|---|---|---|---|
| 1 | 37 | 2400 | 900 |
| 2 | 97.5 | 2400 | 975 |
| 3 | 103.4 | 2400 | 975 |
| 4 | 103.9 | 2400 | 975 |
| 5 | 103.9 | 0 | 250 |

### Example 2

### Treating Ocular Vascular Disorder with Isolated Monocyte Populations

A murine model of oxygen-induced retinopathy was employed to examine therapeutic activities of the monocyte populations isolated with the methods described herein. Mice with oxygen-induced retinopathy were generated as described in Ritter et al., J. Clin. Invest. 116:3266-76, 2006. Specifically, oxygen-induced retinopathy was induced in C57BL/6J mice according to the protocol described by Smith et al., Invest. Ophthalmol. Vis. Sci. 35:101-111, 1994. For comparison, BALB/cByJ mice were also subjected to the same conditions. Briefly, P7 pups and their mothers were transferred from room air to an environment of 75% oxygen for 5 days and afterward returned to room air. The hyperoxic environment was created and maintained using a chamber from BioSpherix. Under these conditions, large hypovascular areas formed in the central retina during hyperoxia in C57BL/6J mice, and abnormal preretinal neovascularization occurred after return to normoxia, peaking at around P17 and ultimately resolving.

Intraocular injection of the isolated cells into the mice was then performed. This is followed by immunohistochemistry analysis and visualization of vasculature in the eyes of the treated mice as well as control mice. These studies were carried out using the procedures described in Ritter et al., J. Clin. Invest. 116:3266-76, 2006. Results from these studies are shown in FIG. 1B. As indicated in the Figure, the substantially pure populations of monocytes isolated by the present inventors were capable of promoting vascular repair in the mice with oxygen-induced retinopathy.

### Example 3

### Other properties and activities of isolated monocyte populations

To demonstrate that the cells we isolated are distinct from other known cell populations in clinical use or development, we have labeled peripheral blood samples for the expression of aldehyde dehydrogenase which, when expressed at high levels (ALDH^{br}), identifies CD34⁺ cells, CD133⁺ cells, kit⁺ cells, Lineage-antigen negative (Lin⁻) cells. We found essentially no such labeling in peripheral blood samples (FIG. 3), fitting with the idea that stem cells are expected to be exceedingly rare in unmobilized peripheral blood.

CD34 is a marker of hematopoietic stem cells and has been used to select cells for various clinical applications. We have found that such cells might comprise or adversely affect the outcome of the therapeutic applications described herein. Specifically, we injected mouse embryonic and human mesenchymal stem cells (which, like CD34⁺ stem cells, are undifferentiated cells) intravitreally in order to determine the behavior of undifferentiated stem cells after intraocular injection. These cells were injected into either normal eyes or those that had undergone the oxygen-induced retinopathy (OIR) model. Additionally, to evaluate the effect of a cell type unrelated to the eye, we intravitreally injected normal human dermal fibroblasts in the mouse OIR model. In all of the above cases, we observed significant inflammatory and neoplastic activity in the retinas. These findings suggest that intraocular injection of undifferentiated stem and/or proliferating cells would lead to significant adverse events in normal or ischemic eyes. These studies also highlight the finding that, in contrast to undifferentiated stem cells, populations of myeloid progenitor cells as described in the present invention, promote a controlled repair of the retinal vasculature without the occurrence of adverse events such as inflammation or neoplasia.

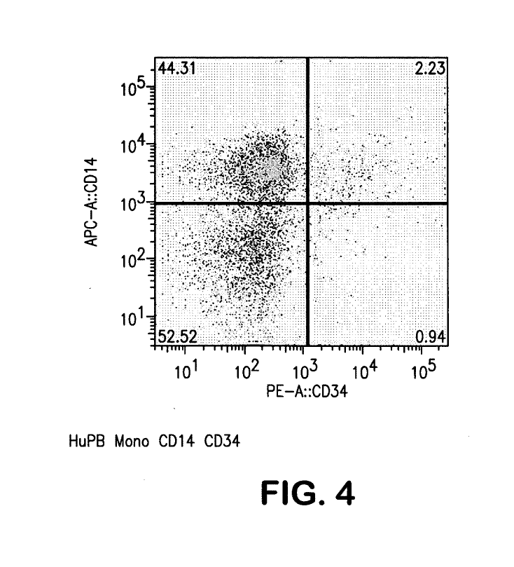

As shown in FIG. 4, the populations prepared using our methods may contain a small number of CD34⁺ cells (FIG. 4). However, these cells are not required for function in our models. In addition, we have specifically depleted CD34-expressing cells from our monocyte preparations and shown no change in efficacy.

### Example 4

### In Vitro Assays for Purity and Function of Isolated Monocytes

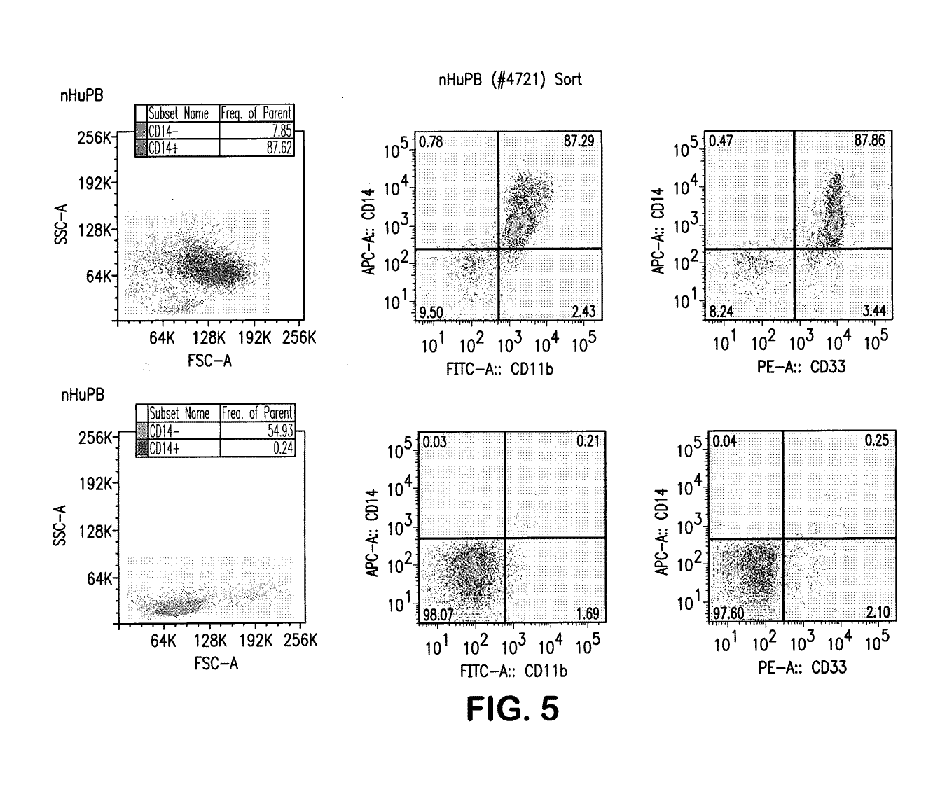

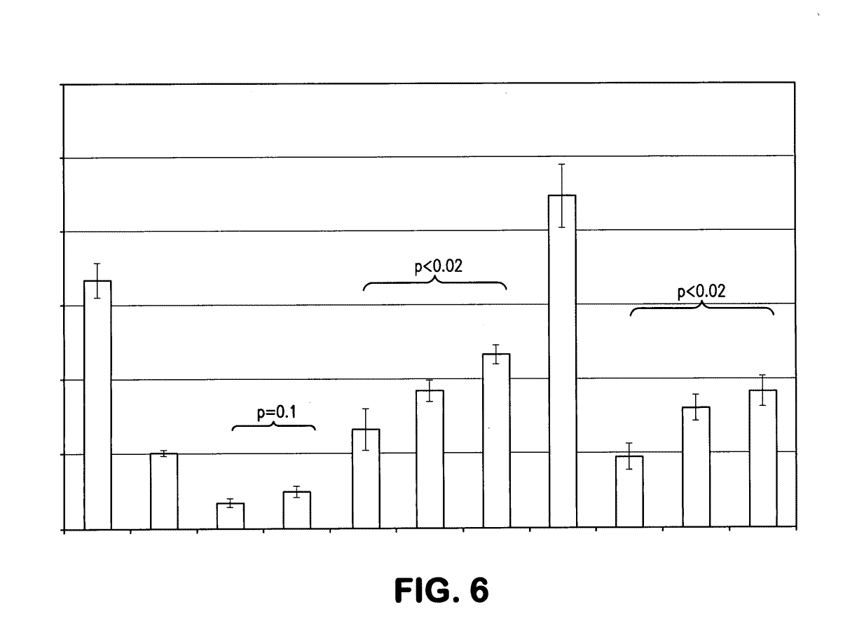

In order to assess the purity and activity of the cells isolated as described above, we developed several in vitro assays that independently evaluate different monocyte characteristics. The first assay was to measure the purity of the monocyte preparation. It used an antibody against the monocyte marker CD14 and flow cytometry (FIG. 5). As shown in FIG. 5, this assay allowed us to determine the number of non-monocyte cells present in the isolated cell population and to validate the efficiency of our isolation methods. The second assay was a measure of the activity of the isolated monocytes. It quantified chemotaxis of cells toward a gradient of monocyte chemotactic protein 1 (MCP-1). These tests were performed using a Boyden chamber with a 3 μm or 5 μm pore size where the cells were allowed to migrate for 2 hrs at 37° C. It was shown that isolated monocyte preparations effectively migrate under the influence of MCP-1, but lymphocytes did not (FIG. 6). Thus, this assay provided a readout of the relative purity of the preparation and an indication of the viability and function of the isolated cells.

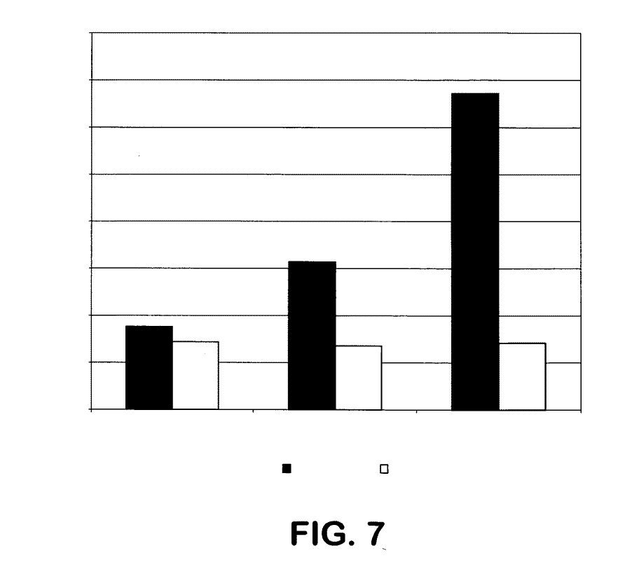

The third assay was based on differential adhesion to cell culture substrata. It is established that monocytes are capable of adhering to cell culture plastic whereas lymphocytes do not adhere. As demonstrated in FIG. 7, results from this assay indicated that the cells generated by the isolation methods described herein were primarily monocytes as evidenced by their ability to adhere under these conditions.

### Example 5

### Systemic Administration of Therapeutic Cell Populations

This Example describes intracardiac administration of CD44^{hi} myeloid cells for therapeutic applications in mouse retinopathy model. This systemic route of delivery differs from the typical local administration route (intraocular injection) used in the above Examples. GFP-expressing CD44^{hi} myeloid cells were prepared and obtained as described in Ritter et al., J. Clin. Invest. 116:3266-76, 2006. Intracardiac injection of the cells into C57BL/6J mice with oxygen-induced retinopathy (typically, postnatal mice at day 7) was performed using standard procedures. Vascular targeting activity of the cells was demonstrated by analyzing GS lectin-stained retinas of the injected mice several days after the injection (e.g., 7 days or 10 days thereafter). Images of the retinal vasculature were obtained using a Radiance2100 MP laser scanning confocal microscope (Bio-Rad; Zeiss). Procedures for staining the retina and analyzing the confocal microscopic images were carried out as described in Ritter et al., J. Clin. Invest. 116:3266-76, 2006.

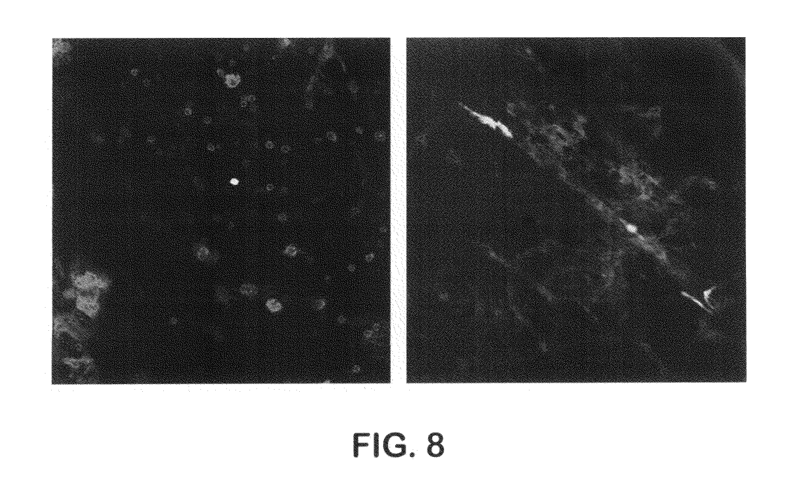

The results obtained from the study demonstrated that a fraction of the therapeutic cells were targeted to the retina after hyperoxic injury (FIG. 8). These findings indicate that the monocyte populations described herein can also be administered systemically (e.g., via intracardiac injection) to achieve their therapeutic effects, e.g., to repair damage or deliver therapeutic agents to the eyes.

### Example 6

### Enhanced Activities of Monocyte Population Activated In Vitro

This Example describes activation of monocyte populations ex vivo and their enhanced activities relative to non-activated cells.

After isolating monocyte cells using the methods described above, the isolated monocyte cells (fraction 5 (F5) cells) were treated with lipopolysaccharide (LPS) at a concentration of 25 ng/ml for 4 hours. Activation was measured through a flow cytometry-based assay, modified from the BD Intracellular Cytokine Staining assay, which measures intracellular levels of cytokines. This assay detected increased accumulation of IL-6, IL-8 and TNF proteins in monocyte (F5) cells that were treated with LPS versus untreated cells and versus lymphocyte-enriched fractions (F3). Specifically, the data showed that while lymphocyte-enriched population (F3) does not substantially activate after LPS, monocyte-enriched population (F5) are clearly activated with LPS. In addition, it was shown that cells derived from diabetic donor activate normally as measured by intracellular cytokine staining. Further, it was found from flow cytometry analysis that LPS treatment has little effect on the morphology of F5 cells as measured by forward scatter vs. side scatter.

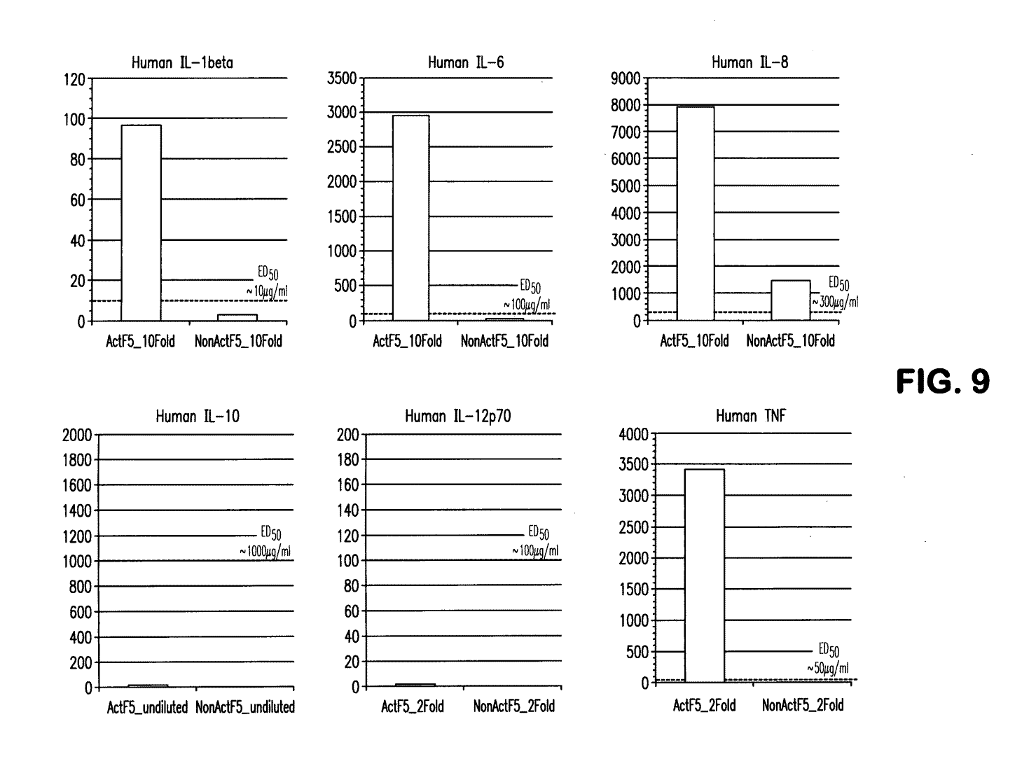

We independently corroborated these findings using a Cytometric Bead Array to quantitatively measure levels of cytokines secreted from LPS-activated cells versus untreated cells. We detected significant increases in secreted IL-6, IL-8 and TNF proteins. This assay also established that IL-1β is significantly upregulated after LPS stimulation in F5 cells (FIG. 9), but secretion of IL-10 and IL-12p70 was essentially unchanged.

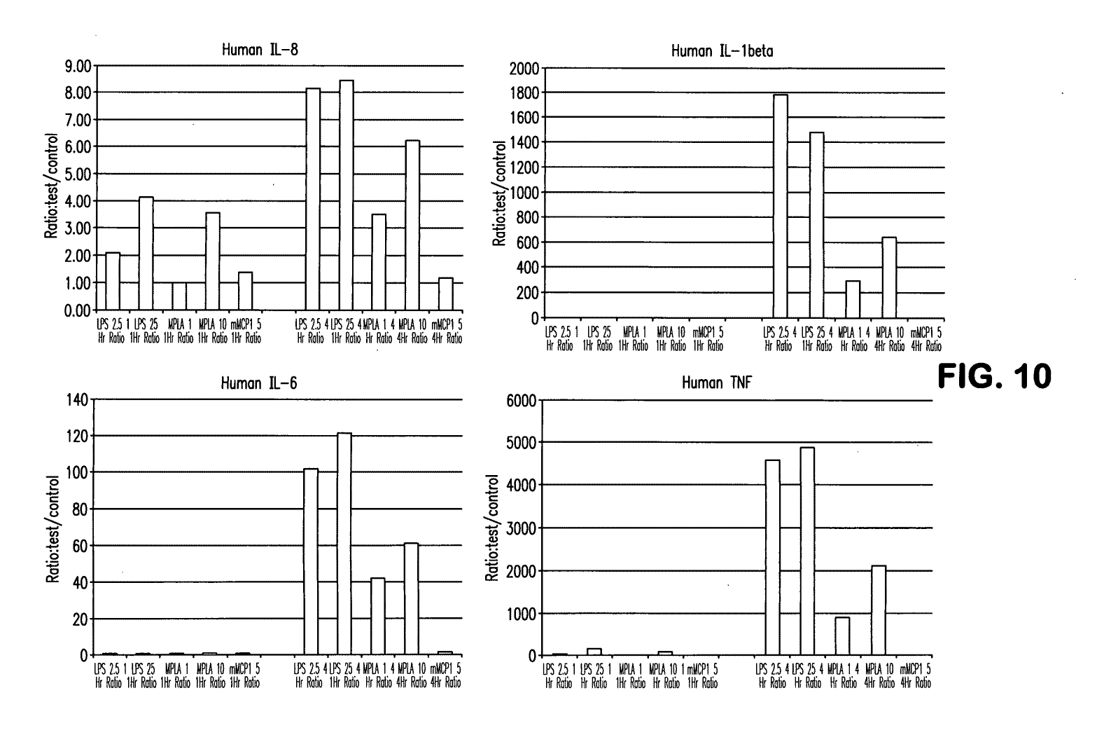

In addition to activating the isolated monocyte cells with LPS, we also examined activities of other activating compounds with more favorable safety profiles. Specifically, we first focused our efforts on alternative ligands for the LPS receptor, TLR4. One of these alternative TLR4 ligands, monophosphoryl lipid A (MPLA), was used in the Cytometric Bead Array described above. The results indicate that MPLA activates F5 cells with increases in IL-8, IL-6 and TNF that were similar to that observed with LPS (FIG. 10). An increase was also observed on IL-10 secretion after MPLA treatment, although the level was approximately half that obtained with LPS stimulation.

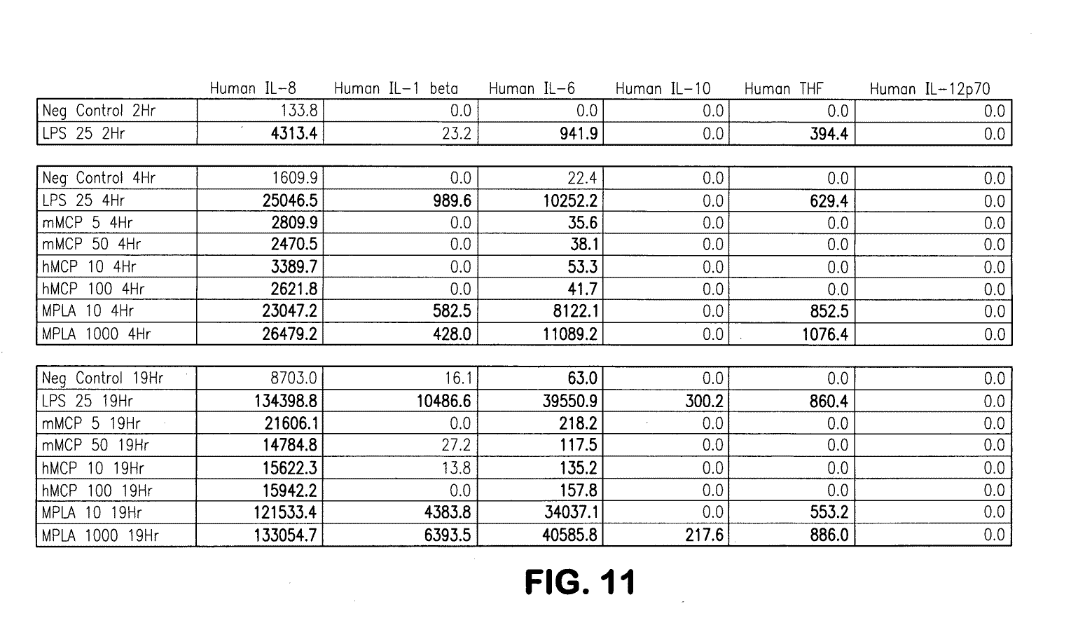

We also tested the activating capacity of mouse and human monocyte chemotactic protein 1 (MCP-1) on F5 monocyte cells. Both mouse and human MCP-1 stimulated increases in IL-8 and IL-6. But the levels were lower than that obtained with LPS or MPLA (FIGS. 10 and 11).

In addition to measuring cytokine secretions of ex vivo activated monocyte populations, we further examined therapeutic activities of the cells in animal studies. In these studies, parallel groups of LPS-treated or control cells were administered to mice via intravitreal injection (250,000 cells in 0.5 μl). These animals were then subjected to hyperoxia and oxygen-induced retinopathy. Analysis of retinas from these animals showed that treatment with F5 monocyte cells activated by LPS reduced the two main parameters measured in this model: area of vaso-obliteration and area of neovascularization (tufts). Reduction in these parameters was greater with LPS-treated F5 than with untreated F5 cells, treated F3 cells, vehicle or LPS alone.

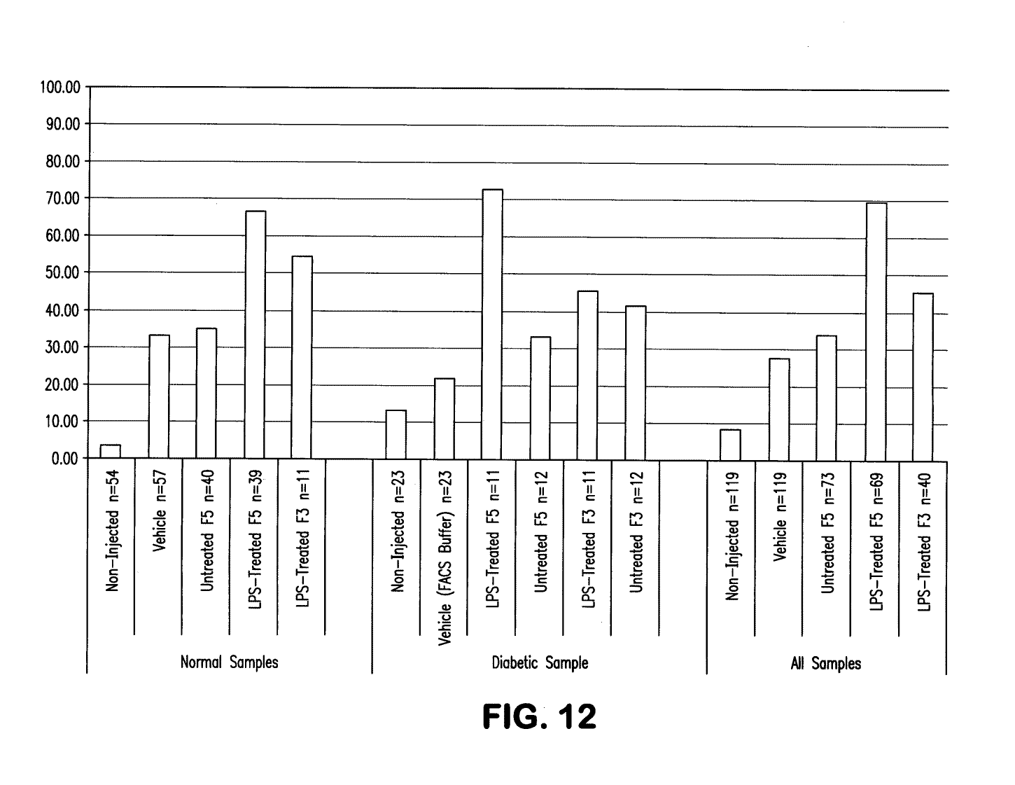

Using a value of 10,000 square microns as a cutoff below which we consider retinas to have essentially no vascular obliteration (described here as “healed”) we were able to demonstrate that a substantially higher number of retinas had areas of obliteration below this cutoff after treatment with LPS-treated F5 cells compared to untreated F5 or other LPS-treated fractions (F3) (FIG. 12). This indicates that activated monocyte-enriched cell populations are capable of promoting vascular repair in this model of ischemic retinopathy. As can be seen from FIG. 12, the F3 fraction shows a level of efficacy in the OIR model, suggesting that active cells are present in this fraction as well. Thus, this population, or a combination of F5 and F3 cells, can also be therapeutically useful.

With the potential use of an autologous approach in the treatment of diabetic retinopathy, it is critical to demonstrate that cells derived from diabetic donors are active. Using the OIR model, we have shown that, in fact, this is the case. Monocyte enriched fractions (F5) from diabetic donors showed an activation pattern that was indistinguishable from normal donors (FIG. 9), and these activated cells were also shown to promote vascular repair in the OIR model to a greater degree than non-activated F5 cells or other LPS-treated fractions (F3) (FIG. 12). Again, some level of activity was observed in the lymphocyte-enriched F3 fraction.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. An isolated cell population comprising substantially pure monocytes that express CD33 antigen and CD14 antigen.

2. The isolated cell population of claim 1, wherein the cell population is isolated from a mammalian peripheral blood sample, a cord blood sample or a bone marrow sample.

3. The isolated cell population of claim 1, wherein cells in the isolated cell population are human cells or murine cells.

4. The isolated cell population of claim 1, wherein at least 70%, 80% or 90% of the cells in the isolated cell population express surface markers CD14 and CD33.

5. The isolated cell population of claim 1, wherein the cell population is CD34⁻.

6. The isolated cell population of claim 1, wherein the cell population is substantially free of ALDH^{br} cells.

7. The isolated cell population of claim 1, which is further activated in vitro.

8. The isolated cell population of claim 7, wherein the isolated cell population is activated with LPS, MPLA, or MCP-1.

9. A method of treating or ameliorating an ocular vascular disorder in a subject, comprising administering to a subject suffering from the ocular vascular disorder an isolated monocyte population, wherein the cell population being administered is in an amount sufficient to treat or ameliorate the ocular vascular disorder.

10. The method of claim 9, where the monocyte population is isolated from a blood sample or a bone marrow sample from the subject.

11. The method of claim 9, where the subject is a human.

12. The method of claim 9, where the monocyte population comprises substantially pure CD14⁺/CD33⁺ cells.

13. The method of claim 9, wherein at least 80% of the cells in the isolated monocyte population are CD14⁺/CD33⁺.

14. The method of claim 9, wherein the ocular vascular disorder is selected from the group consisting of ischemic retinopathy, diabetic retinopathy, retinopathy of prematurity, neovascular glaucoma, central retinal vein occlusions, retina edema, macular degeneration and retinitis pigmentosa.

15. The method of claim 9, wherein the monocyte population is administered to the subject via intravitreal injection.

16. The method of claim 9, wherein the monocyte population is activated in vitro or ex vivo prior to being administered to the subject.

17. The method of claim 16, wherein the monocyte population is activated with LPS, MPLA, or MCP-1.

18. The method of claim 9, wherein the monocyte population is co-administered to the subject with a monocyte-activating compound.

19. The method of claim 18, wherein the monocyte-activating compound is LPS, MPLA, or MCP-1.

20. A method of treating or ameliorating an ocular disease in a subject, comprising (i) isolating from a blood sample or a bone marrow sample of a subject having an ocular vascular disease a substantially pure monocyte population; and (ii) administering the isolated monocyte population to the subject in an amount sufficient to treat or ameliorate the ocular vascular disease, thereby treating or ameliorating symptoms of the ocular vascular disease in the subject.

21. The method of claim 20, where the isolated monocyte population comprises substantially pure CD14⁺/CD33⁺ cells.

22. The method of claim 20, wherein at least about 80% of the cells in the isolated monocyte population express surface markers CD33 and CD14.

23. The method of claim 20, wherein the monocyte population is isolated by (i) debulking red blood cells from the sample; and (ii) separating remaining red blood cells and other cell types in the sample from monocytes based on their size, granularity or density.

24. The method of claim 23, wherein the remaining red blood cells and other cell types are separated from monocytes by density centrifugation or fluorescence-activated cell sorting (FACS).

25. The method of claim 20, wherein the ocular vascular disorder is selected from the group consisting of ischemic retinopathy, diabetic retinopathy, retinopathy of prematurity, neovascular glaucoma, central retinal vein occlusions, macular degeneration and retinitis pigmentosa.

26. The method of claim 20, wherein the isolated monocyte population is activated ex vivo prior to being administered to the subject.

27. The method of claim 26, wherein the monocyte population is activated with LPS, MPLA, or MCP-1.

28. A method of isolating a substantially pure monocyte population, comprising (i) providing a blood sample or a bone marrow sample from a subject; (ii) debulking red blood cells from the sample; and (iii) separating remaining red blood cells and other cell types in the sample from monocytes, thereby isolating a cell population comprising substantially pure monocytes.

29. The method of claim 28, wherein the remaining red blood cells and other cell types are separated from monocytes based on their size, granularity or density.

30. The method of claim 28, wherein the remaining red blood cells and other cell types are separated from monocytes by density centrifugation or fluorescence-activated cell sorting (FACS).

31. The method of claim 28, wherein the other cell types are platelets, granulocytes and granulocytes.

32. The method of claim 28, wherein the red blood cells are debulked by Hespan differential centrifugation or Ficoll density gradient centrifugation.

33. The method of claim 28, further comprising assaying the isolated cell population for expression of surface marker CD14 and CD33.

34. A substantially pure monocyte cell population isolated by the method of claim 28.
